(19) United States
(12) Patent Application Publication
(10) Pub. No.: US 2018/0326381 A1
(43) Pub. Date: Nov. 15, 2018

(54) SYSTEMS AND METHODS FOR PROVIDING A SELF-CONTAINED, VACUUM-PRODUCING MIXING APPARATUS

(71) Applicants: GARY W. BRADLEY, SANTA BARBARA, CA (US); RYAN WARMKE, SANTA BARBARA, CA (US)

(72) Inventors: GARY W. BRADLEY, SANTA BARBARA, CA (US); RYAN WARMKE, SANTA BARBARA, CA (US)

(21) Appl. No.: 15/895,960

(22) Filed: Feb. 13, 2018

Related U.S. Application Data
(60) Provisional application No. 62/505,005, filed on May 11, 2017.

(51) Int. Cl. B01F 15/02; A61B 17/88; B01F 15/00

(52) U.S. Cl. CPC B01F 15/0258; A61B 17/8833; A61B 2017/8838; B01F 15/00448; B01F 15/00006

(57) ABSTRACT

Systems and methods for providing and using a bone cement mixer are described. While the systems can include any suitable component, in some cases, they include a container that is configured to hold an amount of bone cement, a drive shaft coupled to the container, a mixing arm that is coupled to the container and configured to mix the bone cement when the bone cement is disposed within the container, and a vacuum pump configured to draw gas from within the container to form at least a partial vacuum within the container. In some cases, the drive shaft is mechanically coupled to the mixing arm and the vacuum pump such that rotation of the drive shaft forces the mixing arm to move and the vacuum pump to be actuated. Other implementations are also described.

### CROSS-REFERENCE TO RELATED APPLICATION

This non-provisional application claims priority to U.S. Provisional Patent Application Ser. No. 62/505,005 (Attorney Docket No. 27008.2), filed May 11, 2017, and entitled “SYSTEMS AND METHODS FOR PROVIDING A SELF-CONTAINED, VACUUM-PRODUCING MIXING APPARATUS;” the entire disclosure of which is hereby incorporated by reference.

### FIELD OF THE INVENTION

The present invention relates to bone cement. More particularly, some implementations of the described invention relate to systems and methods for providing intra-operative mixing of bone cement through the use of a self-contained, vacuum-producing, bone-cement mixer. While the described systems can include any suitable component, in some cases, they include a container that is configured to hold an amount of bone cement, a drive shaft that is coupled to the container, one or more mixing arms (e.g., paddles, beaters, etc.), a drive mechanism that is configured to convert rotational force provided to the drive shaft by a reamer-driver or other suitable device into movement of the mixing arms and movement of a vacuum pump that is configured to draw gas from within the container to form at least a partial vacuum within the container.

### BACKGROUND AND RELATED ART

Joint replacement surgeries have become more increasingly commonplace. In this regard, hip, knee, shoulder, ankle, and other joint replacement surgeries, as well as bone reconstruction surgeries and reconstructive dentistry, can be effective solutions for reducing pain, increasing mobility, and/or otherwise improving the quality of life of a person who suffers from joint or bone damage.

In many cases, joint replacement and other bone reconstructive surgeries involve coupling a prosthetic implant to a bone and/or filling a space in the bone with a bone cement, such as polymethylmethacrylate (PMMA). In this regard, some bone cements are initially produced as two separate components (e.g., one or more liquids and/or powders) are mixed together at the time of surgery to form the cement. Then, as the cement begins to cure, it is applied to one or more bones, where the cement cures and hardens.

In order to form bone cement, its various components (e.g., powder and/or liquid components) are mixed through a variety of mixing techniques that include mixing the cement manually and/or centrifuging the cement to reduce gas bubbles in the cement. While many conventional techniques and apparatus have been found to be effective at mixing bone cements, such techniques and apparatus are not necessarily without their shortcomings. Indeed, some mixing techniques are prone to allow a mixed cement to have a relatively high volume of gas bubbles, which can weaken the cement. Additionally, some conventional mixing techniques can be relatively labor intensive. Moreover, as many conventional mixing techniques make it difficult for consistent mixing to occur from one batch of cement to another (or even during the mixing of a single batch), such techniques can regularly result in batches of bone cement that have different characteristics (e.g., densities, bubble volume, etc.).

Thus, while systems and methods currently exist that are used to mix bone cement, challenges still exist, including those listed above. Accordingly, it would be an improvement in the art to augment or even replace current techniques with other techniques.

### SUMMARY OF THE INVENTION

The present invention relates to bone cement. More particularly, some implementations of the described invention relate to systems and methods for providing intra-operative mixing of one or more bone cements through the use of a self-contained, vacuum-producing, bone-cement mixer. While the described systems can include any suitable component, in some cases, they include a container that is configured to hold an amount of bone cement. Additionally, in some implementations, the container is used with one or more drive shafts that are coupled to the container, mixing arms (e.g., paddles, beaters, etc.), vacuum pumps that are configured to draw gas from within the container to form at least a partial vacuum within the container, drive mechanisms that are configured to convert rotational force provided to the drive shafts by a reamer-driver or other suitable device(s) into movement of the mixing arms and/or the vacuum pumps. In some cases, the drive shaft is mechanically coupled to the mixing arms and the vacuum pumps such that rotation of the drive shaft forces both the mixing arms to move and the vacuum pump to be actuated.

In accordance with some implementations, the described mixer includes one or more pressure-regulating valves that that are configured to prevent pressure within the container from becoming too low by preventing the pump from continuing to draw gases from within the container once a desired vacuum level has been achieved.

In some implementations, the described mixer includes one or more one-way check valves that are configured to ensure that air does not undesirably enter into the container (and thereby release the vacuum within the container) when the pressure-regulating valves are actuated.

In still other implementations, the mixer comprises one or more pressure-relief mechanisms that allow users to release the vacuum within the container once the cement is mixed so that the container can be opened relatively easily.

In yet other implementations, the mixer comprises one or more torque clutches that are configured to allow the mixing arms and/or pumps to stop actuating (or to reduce their actuation speed) while a portion of the drive shaft (e.g., a portion coupled to a reamer-driver) is allowed to continue to spin.

In even other implementations, the described mixer includes one or more filters (e.g., carbon matrix filters, paper filters, and/or other suitable filters) that are configured to trap and/or otherwise filter out one or more noxious, bad smelling, and/or potentially harmful gases or fumes that are generated within the mixer during cement mixing.

While the methods and processes of the present invention may be particularly useful for providing cement that is configured to be used for attaching a prostheses to a hip joint, a knee joint, a shoulder joint, and/or an elbow joint, those skilled in the art will appreciate that the described systems and methods can be used in a variety of different applications and in a variety of different areas of manufacture. For instance, instead of being used to prepare a bone cement that is used to anchor a prostheses within a hip, knee, shoulder, or elbow joint, the described systems and methods can be used to mix and otherwise prepare bone cement for use in any other suitable field of art, including, without limitation, for use in dentistry, orthodontia, orthopedics, vertebroplasty, kyphoplasty, percutaneous osteoplasty, resection osteoplasty, osteoplasty, bone repair, bone grafting, reconstructive surgery, and/or any other suitable use. Additionally, instead of being used to mix bone cement, the described systems and methods can be used to mix any other suitable material, including, without limitation, one or more epoxies, glues, resins, mortars, foods, edible materials, drinks, paints, coatings, and/or other suitable materials.

These and other features and advantages of the present invention will be set forth or will become more fully apparent in the description that follows and in the appended claims. The features and advantages may be realized and obtained by means of the instruments and combinations particularly pointed out in the appended claims. Furthermore, the features and advantages of the invention may be learned by the practice of the invention or will be obvious from the description, as set forth hereinafter.

### BRIEF DESCRIPTION OF THE DRAWINGS

In order that the manner in which the above-recited and other features and advantages of the present invention are obtained, a more particular description of the invention will be rendered by reference to specific embodiments thereof, which are illustrated in the appended drawings. Understanding that the drawings are not necessarily drawn to scale or in proper proportion, and that the drawings depict only typical embodiments of the present invention and are not, therefore, to be considered as limiting the scope of the invention, the present inventions will be described and explained with additional specificity and detail through the use of the accompanying drawings in which:

FIG. 1 illustrates a perspective, schematic diagram of a self-contained, vacuum-producing, bone-cement mixer in accordance with a representative embodiment of the invention;

FIG. 2 illustrates a perspective, schematic view of the mixer in accordance with a representative embodiment;

FIG. 3 illustrates a top, schematic view of the mixer in accordance with a representative embodiment;

FIG. 4 illustrates a partial, cross-sectional view of the mixer in accordance with a representative embodiment;

FIG. 5 illustrates a partial, cross-sectional view of a portion of a container of the mixer coupled with a plunger in accordance with a representative embodiment;

FIG. 6 illustrates a side, schematic view of the mixer without a cover in accordance with a representative embodiment;

FIG. 7 illustrates a side, schematic view of the mixer in accordance with a representative embodiment;

FIG. 8 illustrates a side, schematic view of the mixer without the cover in accordance with a representative embodiment;

FIG. 9 illustrates a perspective, schematic view of the mixer without the cover in accordance with a representative embodiment;

FIG. 10 illustrates a perspective, schematic view of the mixer in accordance with a representative embodiment;

FIG. 11 illustrates a perspective, schematic view of the mixer without the cover in accordance with a representative embodiment;

FIG. 12 illustrates a side, schematic view of the mixer without the cover in accordance with a representative embodiment;

FIG. 13 illustrates a side, schematic view of the mixer in accordance with a representative embodiment;

FIG. 14 illustrates a side, schematic view of the mixer without the and without a bowl in accordance with a representative embodiment;

FIG. 15 illustrates a top, schematic view of the mixer in accordance with a representative embodiment; and

FIG. 16 illustrates a top, schematic view of the mixer without the cover in accordance with a representative embodiment.

### DETAILED DESCRIPTION

The present invention relates to bone cement. More particularly, some implementations of the described invention relate to systems and methods for providing intra-operative mixing of one or more bone cements through the use of a self-contained, vacuum-producing, bone-cement mixer. While the described systems can include any suitable component, in some cases, they include a container that is configured to hold an amount of bone cement. Additionally, in some implementations, the container is used with one or more drive shafts that are coupled to the container, mixing arms (e.g., paddles, beaters, etc.), vacuum pumps that are configured to draw gas from within the container to form at least a partial vacuum within the container, drive mechanisms that are configured to convert rotational force provided to the drive shafts by a reamer-driver or other suitable device(s) into movement of the mixing arms and/or the vacuum pumps. In some cases, the drive shaft is mechanically coupled to the mixing arms and the vacuum pumps such that rotation of the drive shaft forces both the mixing arms to move and the vacuum pump to be actuated.

As used herein, the term bone cement, cement, and variations thereof may refer to any suitable material that can be mixed in the described mixer, including, without limitation, any biocompatible anchorage and/or reconstructive material that is configured to be disposed between a prosthesis and a bone and/or that can be attached to a bone. In this regard, some non-limiting examples of such cements include one or more polymethylmethacrylates (PMMA or PMMAs), powders (e.g., pre-polymerized) PMMAs, methyl methacrylates (MMA or MMAs), PMMA and/or MMA co-polymer beads and/amorphous powders, radio opacifiers, initiators, liquid MMA monomers, stabilizers, inhibitors, acrylate-based plastics, calcium phosphates, magnesium phosphates, amorphous magnesium phosphates, self-curing cements, self-curing resins, cold-curing cements, dyes, hardening agents, disinfectants, catalysts, and/or any other suitable materials that can be mixed in the described apparatus.

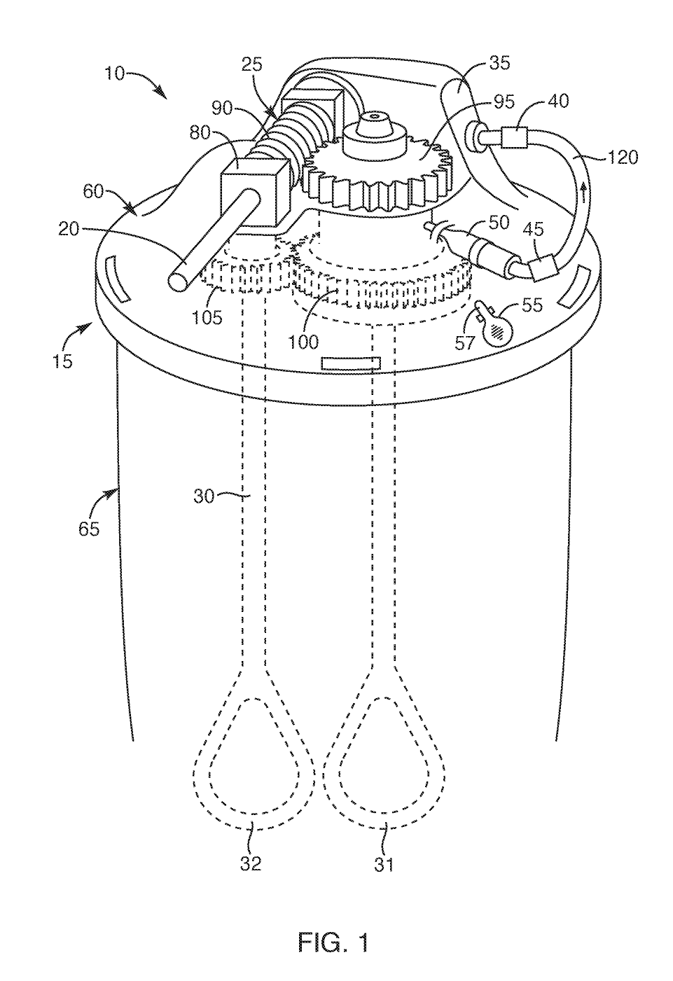

While the described systems can comprise any suitable component, FIG. 1 shows that, in accordance with some representative embodiments, the described self-contained, vacuum-producing, bone-cement mixer 10 optionally comprises one or more containers 15, drive shafts 20, drive mechanisms 25, mixing arms 30, vacuum pumps 35, pressure-regulating valves 40, one-way check valves 45, filters 50, and/or pressure-relief valves 55.

With respect to the container 15, the container can comprise any suitable component that allows it to contain an amount of bone cement and to maintain the cement under a partial vacuum while the cement is mixed (and/or at any other suitable time). Indeed, in some embodiments, the container comprises a first component and a second component (and/or any other suitable number of components) that couple together. By way of non-limiting illustration, FIG. 1 shows an embodiment in which the container 15 comprises a lid 60 and a bowl 65 (i.e., any suitable cement receptacle) that are configured to selectively couple together.

Where the mixer 10 comprises a lid 60 and a corresponding bowl 65, the lid and bowl can couple together in any suitable manner that allows the container 15 to maintain a partial vacuum within the container (e.g., while the cement is mixed within the container and/or at any other suitable time). Indeed, in some embodiments, the lid and bowl are permanently coupled together (e.g., by being formed as a single component, via glue and/or any other suitable adhesive, via welding, and/or in any other suitable manner). In some such embodiments, the container comprises one or more valves, closable outlets, closable inlets, plugs, and/or other suitable mechanisms that allows cement ingredients and/or cement to be added to and/or to be removed from the container.

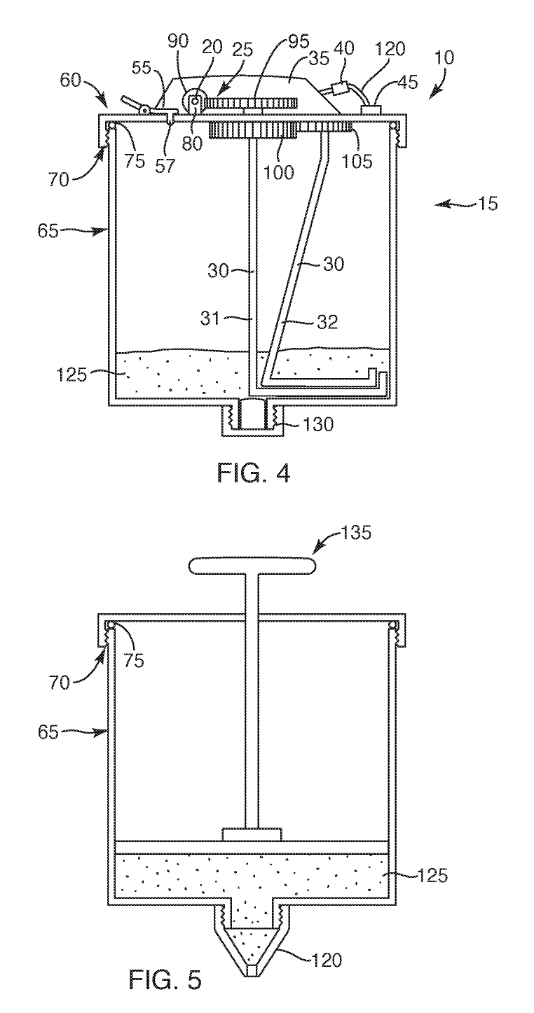

In some other embodiments, however, the lid 60 and bowl 65 are selectively coupleable together and decoupleable from each other. In such embodiments, the lid and bowl can be coupled to and decoupled from each other in any suitable manner, including, without limitation, through the use of one or more threaded engagements, frictional engagements, clamping mechanisms, clamps, seals, sealing mechanisms, gaskets, catches, pins, and/or other suitable mechanisms that allow the container 15 to be selectively opened and/or closed and to maintain a seal and/or partial vacuum when closed (and/or at any other suitable time). By way of non-limiting illustration, FIGS. 1 and 4 show some embodiments in which the lid 60 and bowl 65 are selectively coupleable and decoupleable from each other via threaded engagements 70.

In some embodiments in which the container 15 comprises two or more components (e.g., the lid 60 and the bowl 65) that selectively couple together, one or more gaskets and/or other seals are disposed between such components (e.g., as shown by seal 75 in FIG. 4). In this regard, the container can comprise any suitable seal, including, without limitation, one or more seals comprising one or more rubbers, plastics, papers, cellulose, pieces of carbon, polymers, waxes, clays, polytetrafluoroethylenes, and/or any other suitable materials.

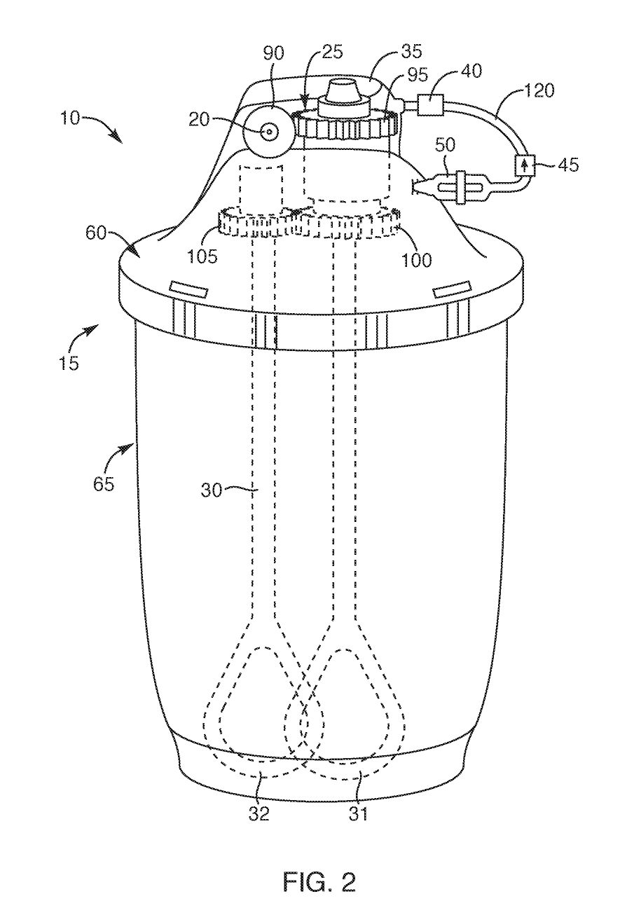

With reference now to the drive shaft 20, the drive shaft can comprise any suitable object that is configured to receive rotational force from an electric, pneumatic, hydraulic, geared, hand crank, and/or other suitable powered reamer-driver, drill, driver, impact wrench, motor, servo, actuator, and/or other powered device (rotary device and/or other suitable device) that is configured to radially spin (and/or otherwise move) the drive shaft. In some embodiments (as shown in FIGS. 1-2), the drive shaft 20 comprises an elongated rod that is rotatably coupled to the container 15 (e.g., via one or more bearings and/or brackets 80) and/or the drive mechanism 25.

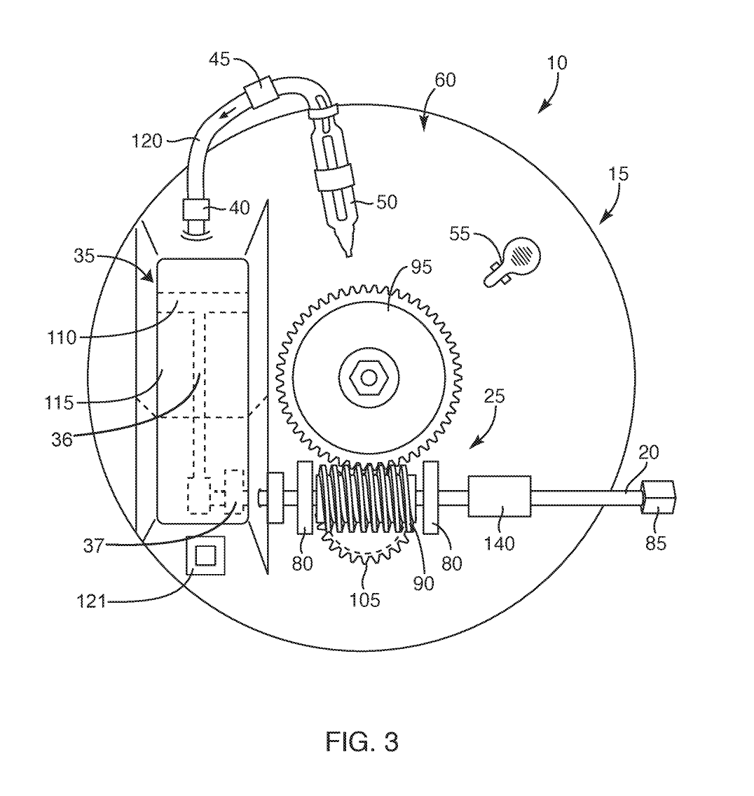

In accordance with some embodiments, one end of the drive shaft 20 is configured to couple with a rotary device (e.g., a reamer-driver) and/or other suitable actuator. In this regard, such end of the drive shaft can have any suitable characteristic, including, without limitation, being cylindrical (e.g., to be clamped in a chuck and/or any other suitable device), being hexagonal, being polygonal, being keyed, being splined, having a socket, being formed to fit in a socket, having a recess for receiving a detent of a quick-release mechanism, having a detent extending from a portion of the shaft, having a quick-connect design that allows the shaft to readily and selectively be coupled to and decoupled from the rotary device, and/or having any other characteristic that allows it to selectively couple to and decouple from a rotary device (and/or other suitable actuator). By way of non-limiting illustration, FIG. 3 shows an embodiment in which the shaft 20 comprises an end 85 that is configured to fit in a socket (e.g., a square, hex, and/or other suitably shaped socket) of a reamer-driver (not shown). In some other embodiments, however, the shaft comprises an end 85 that includes a quick-connect mechanism. For instance, some embodiments of the shaft end comprise one or more recesses that are configured to receive a detent, pawl, and/or other process that is configured to selectively lock the shaft to, and release the shaft from, the rotary device and/or any other suitable actuator.

With respect to the drive mechanism 25, the drive mechanism can include any suitable component that allows the drive mechanism to convert rotational, translational, and/or any other suitable force from the drive shaft 20 into a rotational and/or other movement of the mixing arms 30 that is configured to mix cement within the container 15. Some examples of such mechanisms include, but are not limited to, one or more worms and worm gears, double-enveloping worms and worm gears (or worm drives), bevel gears, spiral-bevel gears, hypoid gears, crown gears, spur gears, meshing gears, universal joints, rack-and-pinion mechanisms, epi-cyclic-gearing systems, sun-and-planet gearing systems, harmonic gears, cage gears, cam mechanisms, gear mechanisms, transmissions, and/or any other suitable mechanisms that are configured to allow the mixer 10 to function as described herein. Indeed, in some embodiments, the drive mechanism is configured convert rotational motion of the drive shaft into mixing (e.g., rotational) motion of the mixing arms. By way of non-limiting illustration, FIG. 1 shows an embodiment in which the drive mechanism 25 comprises a worm 90, a worm gear 95, and meshing gears 100 and 105, wherein the worm 90 is configured to turn the worm gear 95 such that a first mixing arm 31 and/or a first gear 100 are turned and such that a second mixing arm 32 and/or a second gear 105 that intermeshes with the first gear 100 are optionally turned as well.

In accordance with some embodiments, the drive mechanism 25 is configured to move the mixing arms 30 at a slower speed then the speed at which the drive shaft 20 rotates. In this regard, the drive mechanism can down-gear (or otherwise reduce the speed of) the rotation (or other movement) of the mixing arms at any suitable ratio with respect to the rate of rotation of the drive shaft. Indeed, in some embodiments, the mixing arms are configured to rotate (and/or otherwise move) at a ratio that is between about 1:1.5 and about 1:80 times (or any subrange thereof) slower than the rotation of the drive shaft. In other words, in some embodiments, the mixing arms are configured to spin one time for each time the drive shaft rotates between about 1.5 and about 80 times. Indeed, in some embodiments, the mixing arms are configured to make one full rotation for each time the drive shaft makes 20 full rotations (±10 rotations) (e.g., at a ratio of about 1:20±10). Thus, in some embodiments, a user can couple the drive shaft to a rotary device (e.g., a reamer) and spin the rotary device at any speed (e.g., max speed) and the mixer 10 will turn (or otherwise move) its mixing arms at a suitable and substantially constant speed to ensure that cement mixed in the mixer is properly and consistently mixed (e.g., between: different mixers, different batches, different users, etc.).

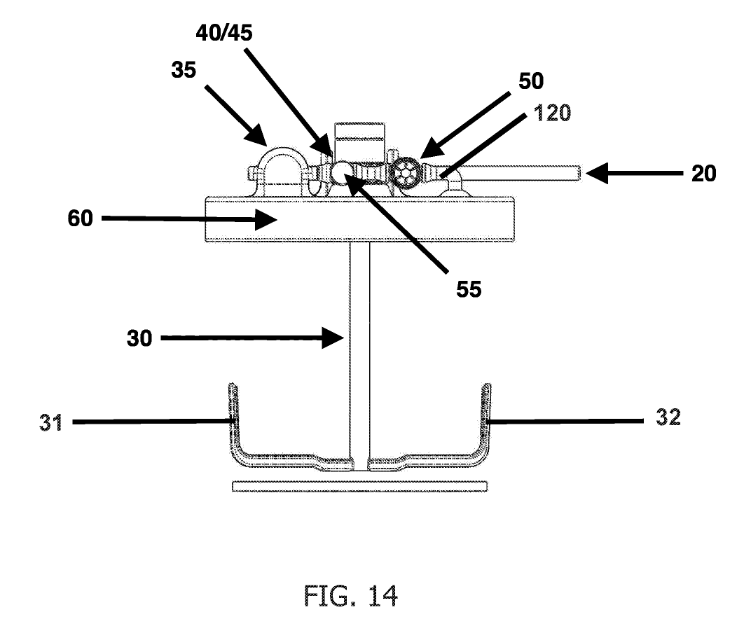

Turning now to the mixing arms 30, the mixer 10 can include any suitable number of mixing arms that allows it to mix cement, including, without limitation, 1, 2, 3, 4, 5, 6, or more. By way of non-limiting illustration, FIGS. 1, 2, and 4 show some embodiments in which the mixer 10 comprises two mixing arms (31 and 32) (see also FIG. 14). Additionally, while the mixing arms can move in any suitable manner, including, without limitation, in a planetary-system method (e.g., as a dough hook on an industrial mixer), in some embodiments (as shown in FIGS. 1, 4, and 14) each mixing arm 30 is configured to rotate in a fixed location.

The mixing arms 30 can comprise any suitable component that allows them to mix bone cement. In this regard, some non-limiting examples of suitable mixing arms include one or more blades, paddles, paddle mixers, arms, spatulas, mixing hooks, whisks, beaters, arms, knifes, whips, scrapers, agitators, and/or other suitable components. By way of illustration, FIGS. 1-2 illustrate some embodiments in which the mixing arms 30 comprise beaters, while FIGS. 4 and 14 illustrate some embodiments in which the mixing arms 30 comprise arms or hooks.

Where the mixer 10 comprises two or more mixing arms (e.g., 31 and 32), the mixing arms 30 can be offset in location, offset via gearing, and/or otherwise configured to be able to spin, rotate, agitate, and/or otherwise move without striking each other and preventing the mixing arms from moving. Indeed, some embodiments of the mixer are configured to prevent the first 31 and second 32 mixing arms from binding against each other during rotation by offsetting the mixing arms in height, location, rotational timing, and/or in any other suitable manner (e.g., as shown in FIG. 4).

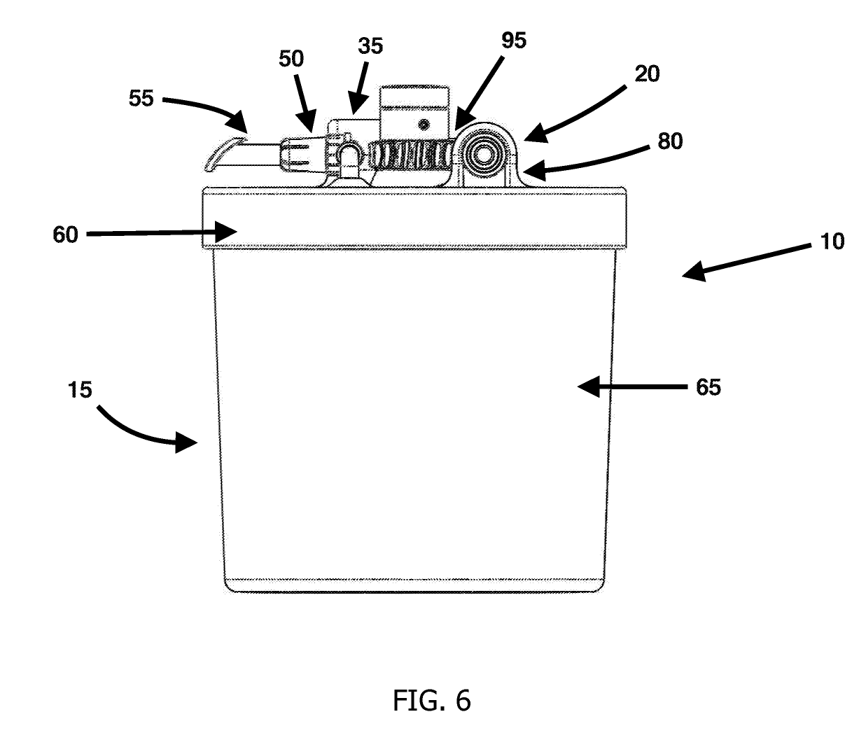

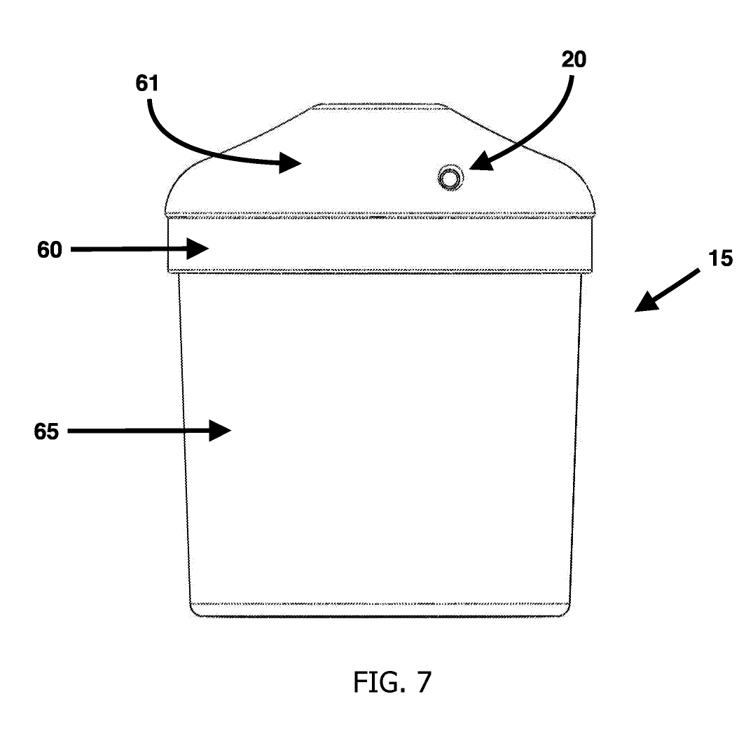

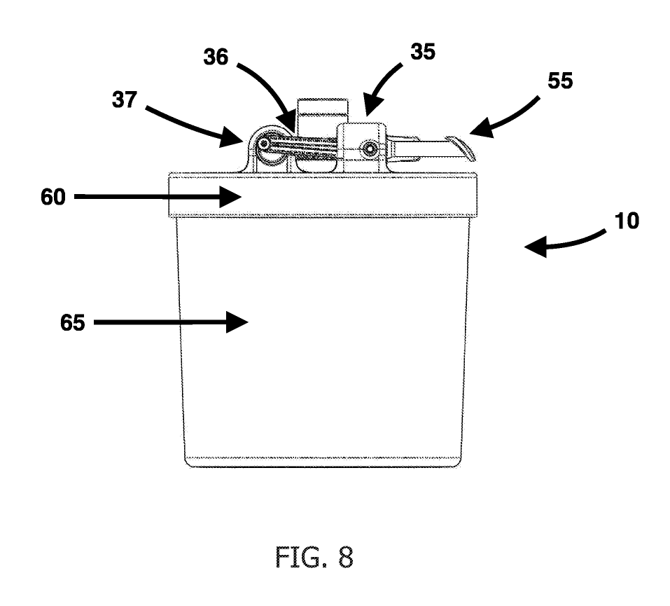

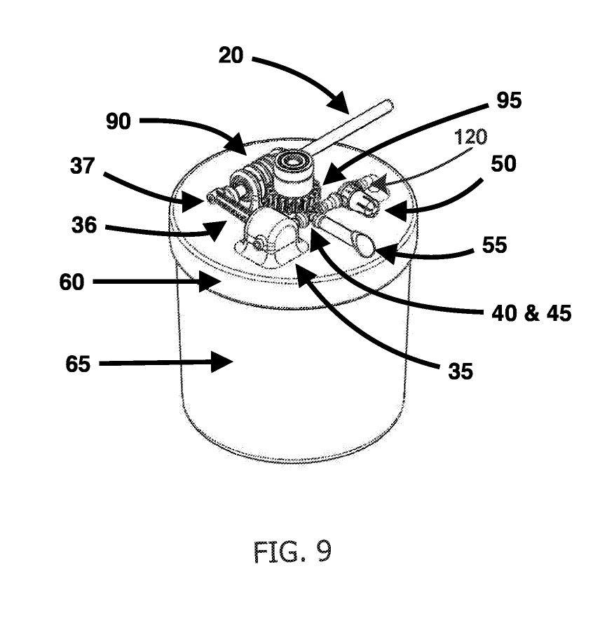

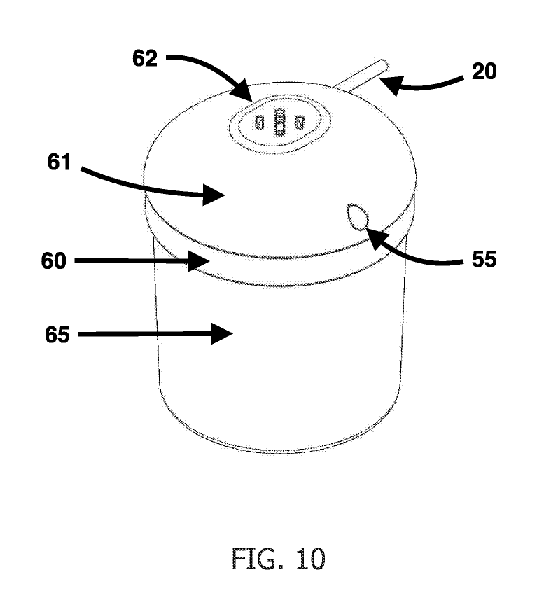

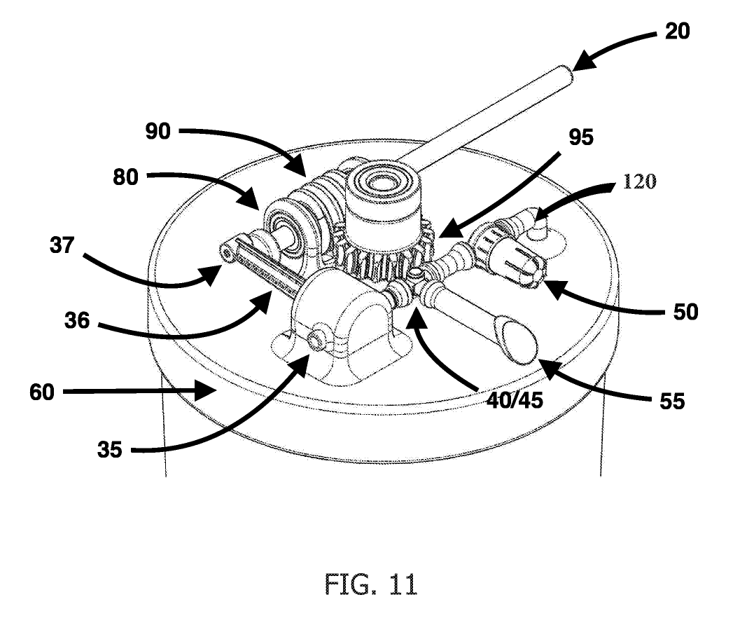

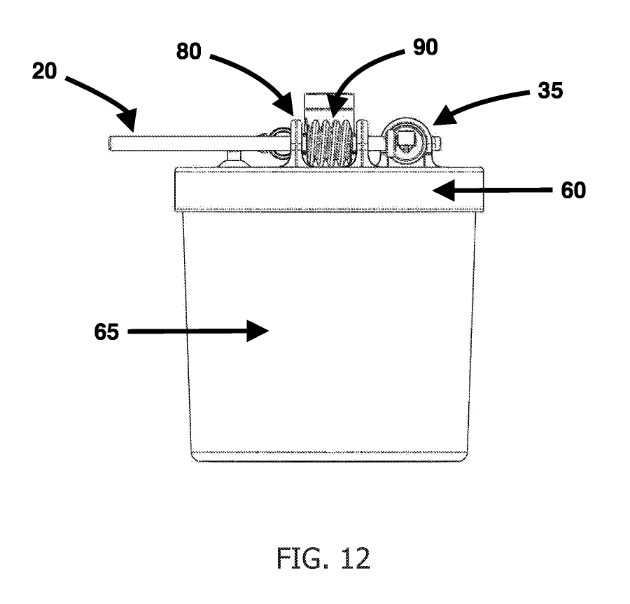

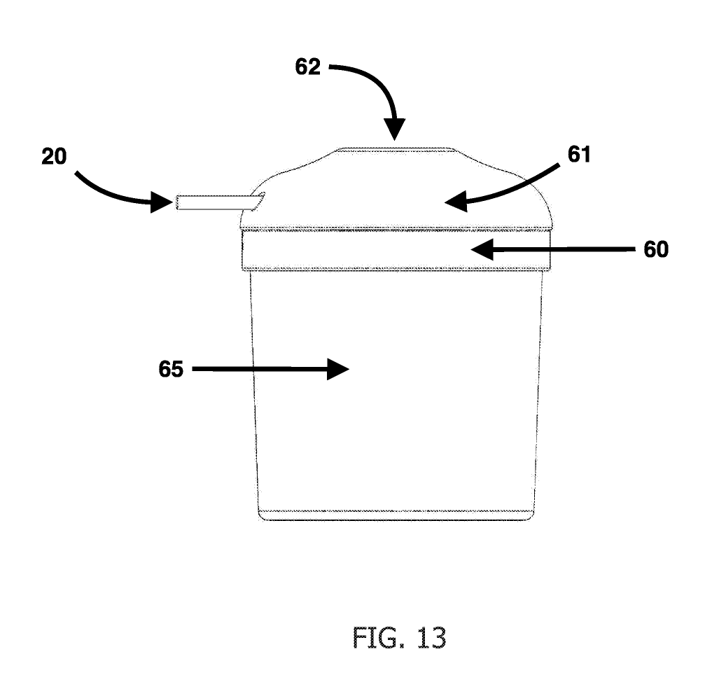

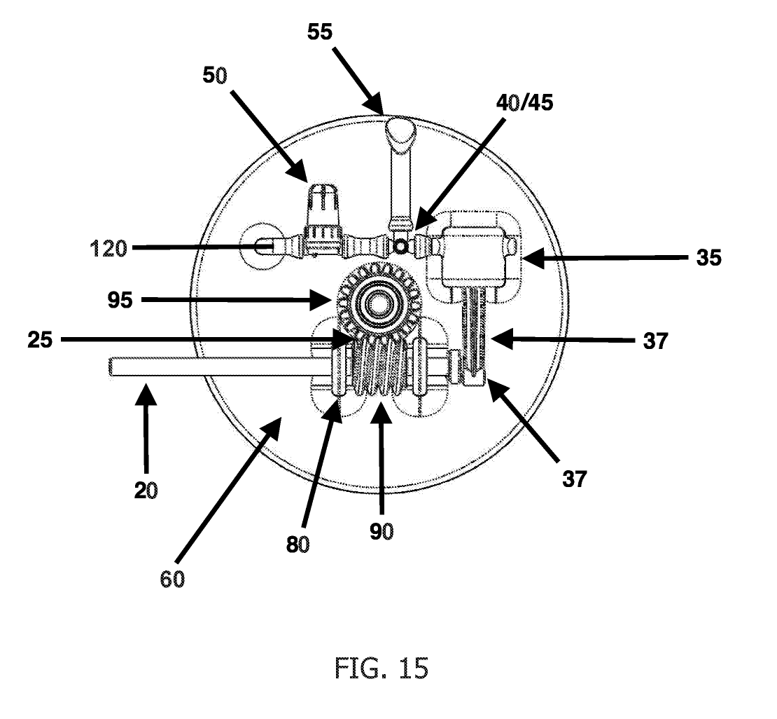

With reference now to the vacuum pump 35, the mixer 10 can be configured to form at least a partial vacuum within the container 15 in any suitable manner and/or via any suitable vacuum creation system. In this regard, some examples of suitable vacuum pumps and other vacuum creation systems, include, but are not limited to, one or more vacuum pumps, centrifugal systems, fans, motorized vacuums, manually-powered vacuum mechanisms, venturi ejectors that use gas and a venturi to draw gases from within the container, external vacuums, and/or other suitable mechanisms that are capable of lowering pressure within the container. By way of non-limiting illustration, FIGS. 1-4, 6, 8, 9, 11, 12, 14, and 15 show some embodiments in which the mixer 10 comprises one or more vacuum pumps 35. In such embodiments, the mixer 10 can comprise any suitable vacuum pump 30 that is configured to create a vacuum (or at least a partial vacuum) within the container. In this regard, some non-limiting examples of such pump mechanisms include one or more diaphragm pumps, fixed displacement piston pumps, axial piston pumps, radial piston pumps, piston pumps, reciprocating pumps, plunger pumps, centrifugal pumps, roots blowers, rotary pumps, and/or other suitable pumps. In accordance with some embodiments, however, the vacuum pump comprises a piston pump.

Where the mixer 10 comprises one or more vacuum pumps 35, the pumps can be driven in any suitable manner, including, without limitation, via rotation of the drive shaft 20, rotation of a hand crank, rotation of a motor on the mixer, pressurized gas, and/or in any other suitable manner. In accordance with some embodiments, however, both the mixing arms 30 and the vacuum pump are actuated by rotation of the drive shaft. By way of non-limiting example, some embodiments of the pump comprise a diaphragm pump that has a moving diaphragm that is configured to remove air from within the container 15 to decrease pressure within the container. In some such embodiments, the drive shaft continues past the drive mechanism 25 (e.g., worm 90) to drive the vacuum pump. Additionally, in some such embodiments, a portion of the shaft comprises and/or is coupled with a cam, an offset connecting rod, an eccentric wheel, and/or any other suitable component that attaches to a piston (e.g., piston 110, shown in FIG. 3) contained in a cylinder (e.g., cylinder 115, shown in FIG. 3). Indeed, FIGS. 3 and 8 show that in some embodiments, the drive shaft 20 and drive mechanism 25 are configured to turn a cam 37 that is coupled to a piston rod 36, which in turn is coupled to a piston 110. In some such embodiments, as the piston moves, gases that are pulled from an intake port that opens into the container (e.g., via a tube 120 or otherwise) are optionally passed through one or more filters (e.g., filter 50) and are then expelled through one or more exhaust ports (e.g., exhaust vents 121 and/or 62; see e.g., FIGS. 3, 10, and 13).

With reference now to the pressure-regulating valve 40, some embodiments of the of the mixer 10 optionally comprise one or more shut-off valves, vacuum-relief valves, one-way filters, one-way valves, and/or pressure-regulating valves or mechanisms that are configured to prevent the vacuum pump 35 from continuing to draw gases from within the container 15 once a desired pressure (or vacuum) has been reached within the container. By way of non-limiting illustration, FIGS. 1-4, 9, 11, 14, and 15 show that some embodiments of the mixer 10 comprise one or more pressure-regulating valves 40 that are configured to allow the vacuum pump 35 to draw air (e.g., ambient air and/or any other suitable gas) through the pump and to prevent the pump from continuing to draw gases from within the container once a desired vacuum level has been achieved. In this regard, while the pressure-regulating valve can be disposed in any suitable location, FIGS. 1-4, 9, 11, 14, and 15 show some embodiments in which the pressure-regulating valve 40 is coupled to a conduit 120 that extends between the vacuum pump 35 and the container 15.

Where the mixer 10 comprises one or more vacuum pumps 35 and/or pressure-regulating valves 40, the mixer can be configured to achieve and/or maintain any suitable pressure or vacuum level within the container 15 (e.g., to reduce bubbles in the cement and/or for any other suitable purpose). Indeed, in some embodiments, the mixer is configured to obtain a negative pressure (or vacuum level) within the container that is between about 10 mm hg and about 1,150 mm hg (or any subrange thereof) from (or below) ambient pressure as cement is mixed within the mixer (and/or at any other suitable time). Indeed, in accordance with some embodiments, the pressure-regulating valve is configured to activate once pressure within the container reaches a set pressure (or vacuum level) between about 400 mm hg and about 650 mm hg (e.g., between about 500 mm hg and about 550 mm hg) from or below ambient pressure.

With reference now to the one-way check valve 45, some embodiments of the mixer 10 comprise one or more one-way check valves that are configured to ensure that air is not drawn through the vacuum pump 35 into the container 15, including, without limitation, when the pressure-regulating valve 40 is engaged. Accordingly, in some embodiments, the one-way check valve can ensure that the desired vacuum level is maintained within the container when the pressure-regulating valve is actuated. In this regard, the check valve can be disposed in any suitable location on the mixer 10. In some non-limiting illustrations, however, FIGS. 1-4, 9, 14, and 15 show some embodiments in which the check valve 45 is disposed between the container 15 and the pressure-regulating valve 40.

Referring now to the filter 50, some embodiments of the mixer 10 optionally comprise one or more filters. In this regard, the filters can perform any suitable function. Indeed, as some types of cements may produce noxious (or otherwise undesirable fumes) during mixing, some embodiments of the filter are configured to filter (and/or remove some of the noxious fumes from) the gases that are drawn by the pump out of the container 15 and into ambient air. In this regard, the mixer can comprise any suitable filter that is configured to perform such a function, including, without limitation, one or more charcoal, activated-carbon, fiberglass, paper, foam, cotton, high efficiency particulate air, and/or any other suitable filters. Indeed, in accordance with some embodiments, the filter comprises a charcoal filter (e.g., a carbon matrix filter and/or any other suitable carbon filter).

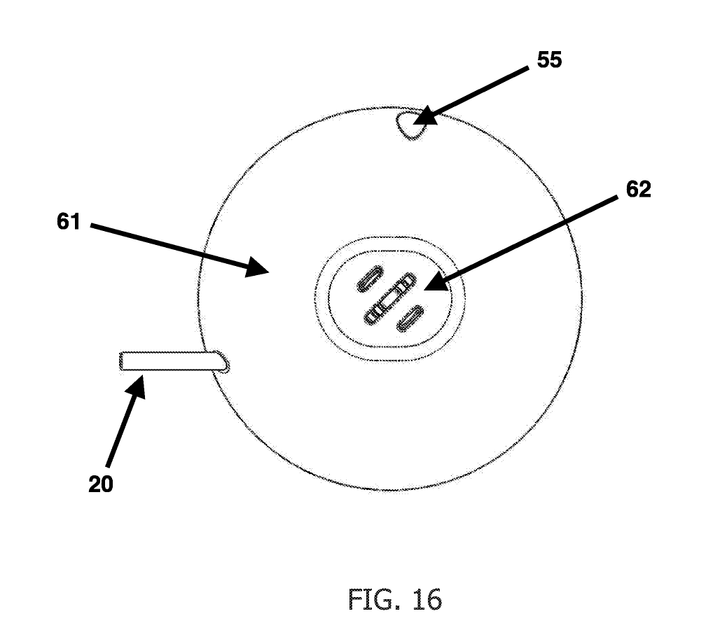

Turning now to the pressure-relief valve 55, some embodiments of the mixer 10 optionally comprise one or more pressure-relief valves that allow the partial vacuum within the container 15 to be released (or at least partially released) before or when the container is opened (e.g., to make it easier to open the container). In this regard, while the pressure-relief valve 55 can be disposed in any suitable location, FIGS. 1-4, 6, 8-11, and 14-16 show some embodiments in which it is disposed in the lid 60. Additionally, while the pressure-relief valve can (but does not necessarily) comprise a spring-loaded plunger with a seal that is configured to be moved to allow air to enter into the container to relieve a vacuum in the container, a removable plug, and/or any other suitable mechanism, FIG. 4 illustrates an embodiment in which the pressure-relief valve 55 comprises a lever-actuated stopper 57 that is configured to be selectively removed from and returned to an opening in the lid 60.

In addition to the aforementioned characteristics, the described mixer 10 can comprise any other suitable component that allows it to be used to mix bone cement (and/or any other suitable material, as noted herein). Indeed, in some embodiments, the mixer comprises a cover that is configured to extend over one or more components of the mixer (e.g., a portion of: the drive shaft 20, the drive mechanism 25, the vacuum pump 35, and/or any other suitable component). In this regard, the cover can perform any suitable purpose, including, without limitation, serving to prevent gloves, fingers, and/or other items from being caught by the mixer and/or improving the aesthetic appearance of the mixer.

Where the mixer 10 comprises a cover, the cover can be disposed in any suitable location, including, without limitation, on the lid 60, the bowl 65, and/or in any other suitable location. By way of non-limiting illustration, FIGS. 7, 10, 13, and 16 show some embodiments in which a cover 61 is coupled to the lid 60. In such embodiments, the cover can couple to the lid in any suitable manner, including, without limitation, via one or more friction fittings, fasteners, glues, epoxies, cements, clamping mechanisms, welds, and/or other suitable mechanisms.

As another example of a suitable component or characteristic, in some embodiments, the mixer comprises or is configured to attach (e.g., via a friction fitting, a threaded engagement, a mechanical engagement, and/or in any other suitable manner) with one or more nozzles that are configured to deliver cement from within the container 15 to a desired location (e.g., a bone). In this regard, while such a nozzle can be disposed in any suitable location (e.g., on a side, bottom, top, and/or any other suitable location) of the container 15, FIGS. 4 and 5 show that in some embodiments, the nozzle 120 is disposed at a bottom end of the container 15 (e.g., bowl 65). Specifically, FIGS. 4-5 show that, in some embodiments, once the cement 125 is mixed in the bowl 65, a cap/stopper 130 and/or the lid 60 can be removed from the bowl, and the nozzle 120 and/or a plunger assembly 135, an auger assembly, a screw assembly, and/or any other suitable assembly that is configured to be used to force cement from the container can be coupled to the bowl and be used to force the cement from the bowl (e.g., through the nozzle or otherwise).

Where the mixer 10 is configured to be used with a plunger assembly 135, the plunger assembly can comprise any suitable component that allows it to function as described herein (e.g., a plunger, a seal formed against the plunger and the bowl, and/or any other suitable component). In some embodiments, the plunger assembly is configured to function much like a medical syringe (e.g., as illustrated in FIG. 5). In some other embodiments, the plunger assembly comprises a ratchet mechanism that is configured to ensure that a plunger of the assembly is able to retain its position (even if only selectively) as it is advanced within the bowl 65. In still other embodiments, the plunger assembly is configured to be actuated via a trigger mechanism, a plier actuated mechanism, a lever mechanism (e.g., like a caulking gun or otherwise), a screw mechanism, and/or in any other suitable manner.

As another example of a suitable modification, some embodiments of the mixer 10 comprise a thermometer (or other indicator) that is configured to indicate when the cement has a reached a suitable temperature (e.g., to determine the state of polymerization) for application to a bone. In this regard, while the mixer can comprise any suitable indicator that is capable providing such an indication, in some embodiments, the mixer comprises a sticker thermometer or temperature indicator.

In still another example of a suitable modification, the rotary device, another motor or device that is configured to turn the drive shaft 20, and/or the mixer itself comprises a timer that is configured to automatically slow, shut off, and/or otherwise control the rotary device (or the mixing arms 30 and/or the vacuum pump 35) once the rotary device has mixed the cement for a predetermined amount of time.

In another example, some embodiments of the mixer 10 are configured (e.g., via a clutch, a torque clutch, a weak motor, by overloading the motor, and/or in any other suitable manner) to stop mixing cement when a consistency (or viscosity) of the cement reaches a set level (e.g., as the cement thickens).

Indeed, some embodiments of the mixer 10 comprise a torque clutch that is configured to allow the speed (and/or the ratio) at which the mixing arm or arms 30 turn (or are otherwise moved) to vary with respect to the speed at which the drive shaft 20 turns. Indeed, in some embodiments, when movement of the mixing arms 30 is hindered (e.g., as the cement thickens, as the mixing arms hit a lump in the cement, and/or as the mixing arms otherwise slow), the torque clutch is able to “slip”, thus allowing the drive shaft (or a portion thereof) to spin while the mixing arms' speed slows or even stops. In this regard, while the torque clutch can be disposed in any suitable location on the mixer, FIG. 3 shows some embodiments in which the torque clutch 140 is disposed on the drive shaft 20.

In still another example of a suitable modification, in some embodiments, the drive shaft 20 is coupled (or is configured to be coupled) to a hand crank that is configured be manually rotated to operate the mixer (e.g., the mixing arms 30 and/or the vacuum pump 35).

In even another example of a suitable modification, some embodiments of the mixer 10 comprise one or more motors and/or batteries that are disposed on the mixer and that are capable of operating the mixer without the use of separate rotary device. In some cases, such components are coupled directly to the mixer (e.g., on the lid 60 and/or in any other suitable location). In some other embodiments, a component comprising a motor and/or power source is configured to be coupled to the mixer (e.g., placed on the lid and/or in any other suitable location). Thus, in some embodiments, a motorized component (a motorized component that is tailored specifically for the mixer) is selectively coupleable to and decoupleable from the mixer.

In an additional example of a suitable modification, some embodiments of the mixer 10 comprise one or more self-contained venturi devices (or venturi devices that are used in connection with the mixer). In some such embodiments, the mixer further comprises one or more pressurized tanks comprising a gas (e.g., nitrogen, carbon dioxide, and/or any other suitable gas or gases) that can be used with the venturi to reduce pressure within the mixer. Indeed, in some embodiments, the mixer comprises a mini pressurized gas canister (e.g., a reusable and/or disposable canister) that is used with the venturi to create a vacuum within the container 15.

In another example, a volume of the container 15 is configured to be expanded (e.g., to reduce pressure within the container) once cement ingredients have been added to the mixer (and/or at any other suitable time). In this regard, the volume of the container can be increased in any suitable manner. Indeed, in some embodiments, the mixer comprises one or more pistons (not shown) that are disposed and form a seal within the container and that can be extracted to increase a volume of a portion of the container that includes the cement (e.g., to create a partial vacuum around the cement).

In still another example, in place of and/or in addition to the vacuum pump 35, some embodiments of the mixer 10 comprise a pump that is configured to be actuated (e.g., via the drive shaft 20, drive mechanism 25, manually, and/or in any other suitable manner) to increase a pressure within the container 15. In this regard, such a pump can be used for any suitable purpose, including, without limitation, to force cement from the mixer. Thus, the mixer can comprise any suitable type of pump that is configured to increase pressure within the container, including, without limitation, one or more diaphragm pumps, fixed displacement piston pumps, axial piston pumps, radial piston pumps, piston pumps, reciprocating pumps, plunger pumps, centrifugal pumps, roots blowers, rotary pumps, and/or other suitable pumps.

As yet another example of a suitable modification, although some embodiments of the mixer 10 are configured to be held in a user's hand as the cement is mixed in the mixer, some other embodiments of the mixer are configured to be coupled to an object (e.g., via one or more catches, clamps, suction cups, frictional engagements, mechanical couplers, and/or any other suitable device that is configured to hold the mixer) while the mixing arms 30 mix the cement. Thus, in some embodiments, the mixer can be operated relatively easily and safely.

The various components of the described mixer 10 can be made from any suitable material, including, without limitation, one or more: types of plastics (e.g., polyvinyl chloride, polypropylene, polyethylene, polystyrene, nylon, polyethylene terephthalate, polyimide, polycarbonate, acrylonitrile butadiene, polyetheretherketone, polyurethane, and/or any other suitable plastic or polymer), metals (e.g., stainless steel, titanium, titanium-based alloys, tantalum, cobalt-based alloys, and/or any other suitable metal or metal alloy), ceramics, types of glass, synthetic materials, natural materials, and/or other suitable materials. Indeed, in some embodiments, the drive shaft 20 comprises a metal, while several other components of the mixer (e.g., the lid 60, the bowl 65, etc.) comprise plastic, such that the mixer can be sterilized, packaged, used, and/or discarded. In some other embodiments, however, one or more portions of the mixer comprise metal and/or any other suitable material that allows such portions to be autoclaved and reused.

The various portions of the described mixer 10 can be made in any suitable manner. In this regard, some non-limiting examples of methods for making the described mixer include molding; extruding; boring; shaping; machining; etching; cutting; bending; drilling; grinding; sanding; lathing; smoothing; buffing; polishing; connecting various pieces with one or more adhesives, mechanical fasteners (e.g., nails, clamps, rivets, staples, clips, pegs, crimps, pins, brads, threads, brackets, etc.), welds, and/or by melting pieces together; and/or any other suitable method that allows the described system to perform its intended functions.

The described mixer 10 can be used in any suitable manner. In this regard, while one embodiment of a method for using the mixer is described herein, such method can be modified in any suitable manner, with various portions of the method being omitted, added to, performed in a different order, substituted, performed simultaneously, performed in series, and/or otherwise modified from the described method. In any case, in some embodiments, the mixer is used as one or more cement ingredients are added into the bowl 65, the lid 60 is coupled with the bowl (e.g., by twisting the lid and bowl together or in any other suitable manner), and a rotary device is coupled to the drive shaft 20. With the rotary device coupled to the drive shaft, the drive shaft can then be rotated at any suitable rate (e.g., between about 1 rpm and about 4,000 rpm, or within any subrange thereof). Indeed, in some embodiments, the rotary device is spun at its maximum speed (e.g., between about 20 rpm and about 2,400 rpm) for any suitable amount of time (e.g., between about 1 second and about 15 minutes, or any sub-range thereof (e.g., between about 15 seconds and about 90 seconds)).

In accordance with some embodiments, as the drive shaft 20 rotates, the drive shaft actuates the drive mechanism 25 (e.g., the worm 90 on the drive shaft actuates the worm gear 95), which in turn operates the mixing arms 30 and mixes the ingredients to form bone cement. Additionally, in some embodiments, as the drive shaft rotates, the shaft further causes the vacuum pump 35 to actuate—drawing gases out the container 15, through a filter 50, and expelling the gases through an exhaust vent (or port) 121 and/or 62.

In some embodiments, once an appropriate vacuum level is achieved in the container 15, the pressure-regulating valve 40 is actuated to prevent the pressure in the container 15 from dropping further, and the one-way check valve 45 prevents gases from undesirably flowing back into the container.

In some cases, once the cement 125 has been mixed for a suitable amount of time, mixing is stopped (e.g., by stopping the rotary device and/or otherwise), the rotary device is removed from the shaft 20, the vacuum in the container is optionally released (e.g., via the pressure-relief valve 55), the lid 60 is removed from the bowl 60, and the cement is ready for application (e.g., via the plunger assembly 135 and nozzle 120, removal from the container 15 with a spatula and/or any other suitable object, and/or in any other suitable manner).

In addition to the aforementioned features, the described mixer 10 can comprise any other suitable feature. Indeed, unlike some conventional bone-cement-mixing apparatus that include a venturi ejector that is driven by compressed nitrogen (and/or another inert gas), and as such comprise one or more tubes that carry nitrogen from a nitrogen repository (e.g., a nitrogen tank or other gas delivery system) to such apparatus, some embodiments of the described mixer do not require that any cumbersome tubes or wires be run to the mixer in order for the mixer to function. As a result, some such embodiments of the described mixer can be used without tubes or wires getting in the way and/or the worry that such tubes may become contaminated and may thereby pass contamination in the operating room. Additionally, as some embodiments of the described mixer do not require compressed nitrogen, such embodiments do not require the storage, replenishment, cost, and/or maintenance of a pressurized nitrogen source.

As another example of a beneficial feature, some conventional bone-cement-mixing apparatus are configured to be actuated by hand and, as a result, may easily and inadvertently be used to mix cements at different speeds, allowing cements produced by such systems to have varied characteristics (e.g., densities, bubble volumes, etc.). In contrast, some embodiments of the described mixer 10 are configured to easily allow users to mix batches of cement (e.g., with a different mixer, in the same mixer, or otherwise) at substantially the same mixing speed and under substantially the same mixing conditions. Indeed, in some embodiments, by spinning a rotary device (e.g., a reamer, a drill, etc.) that is coupled with the drive shaft 20 at full speed (and/or any other desired set speed), one or more users can mix many different batches of cement (in the same or in a different mixer), with each batch having been mixed at substantially the same speed and thus having substantially similar characteristics (e.g., bubble volume, density, etc.).

As another example of a feature of the described mixer 10, some embodiments of the described system are configured to effectively produce a desired vacuum level within the container 15. Accordingly, such embodiments of the described systems and methods can produce relatively dense cement having relatively small amounts of bubbling without the need for a centrifuge, which can often provide inconsistent cement densities and require a large, heavy, expensive, and time-consuming device. In this regard, it has been found that, in some cases, stronger bone cement is produced when gas bubbles and inclusions (like blood) in the cement are decreased.

As still another example of a feature of some embodiments of the mixer 10, one or more portions of the described mixer are disposable. Indeed, while, in some embodiments, some portions of the described apparatus are autoclavable (e.g., the bowl 65, the lid 60, the drive shaft 20, the vacuum pump 35, and/or any other portion of the mixer 10), in some other embodiments, the entire mixer is configured to be discarded after a single use.

In still another example, some embodiments of the described mixer are configured to function using only instruments (e.g., a reamer-driver, drill, etc.) that are typically available for use in a sterile surgical field.

Thus, as discussed herein, embodiments of the present invention relate to bone cement. More particularly, some implementations of the described invention relate to systems and methods for providing intra-operative mixing of bone cement through the use of a self-contained, vacuum-producing, bone-cement mixer. While the described systems can include any suitable component, in some cases, they include a container that is configured to hold an amount of bone cement, a drive shaft that is coupled to the container, one or more mixing arms (e.g., paddles, beaters, etc.), a drive mechanism that is configured to convert rotational force provided to the drive shaft by a reamer-driver or other suitable device into movement of the mixing arms and a vacuum pump that is configured to draw gas from within the container to form at least a partial vacuum within the container. In some cases, the drive shaft is mechanically coupled to the mixing arms and the vacuum pump such that rotation of the drive shaft forces both the mixing arms to move and the vacuum pump to be actuated.

The present invention may be embodied in other specific forms without departing from its spirit or essential characteristics. Additionally, one or more components from any of the described embodiments and examples are interchangeable with any other components of the other described embodiments and examples. The described embodiments, examples, and illustrations are to be considered in all respects only as illustrative and not restrictive. The scope of the invention is, therefore, indicated by the appended claims rather than by the foregoing description. All changes that come within the meaning and range of equivalency of the claims are to be embraced within their scope. In addition, as the terms on, disposed on, attached to, connected to, coupled to, etc. are used herein, one object (e.g., a material, element, structure, member, etc.) can be on, disposed on, attached to, connected to, or coupled to another object—regardless of whether the one object is directly on, attached, connected, or coupled to the other object, or whether there are one or more intervening objects between the one object and the other object. Also, directions (e.g., front back, on top of, below, above, top, bottom, side, up, down, under, over, upper, lower, lateral, etc.), if provided, are relative and provided solely by way of example and for ease of illustration and discussion and not by way of limitation. Where reference is made to a list of elements (e.g., elements a, b, c), such reference is intended to include any one of the listed elements by itself, any combination of less than all of the listed elements, and/or a combination of all of the listed elements. Furthermore, as used herein, the terms a, an, and one may each be interchangeable with the terms at least one and one or more.

What is claimed is:

1. A bone cement mixer comprising:
a container configured to hold an amount of bone cement;
a drive shaft coupled to the container and configured to couple with a powered rotary device;
a first mixing arm coupled to the container and configured to mix the bone cement when the bone cement is disposed within the container; and
a vacuum creation system configured to draw gas from within the container to form at least a partial vacuum within the container,
wherein the drive shaft is mechanically coupled to the first mixing arm such that rotation of the drive shaft forces the first mixing arm to rotate.

2. The mixer of claim 1, further comprising a second mixing arm, wherein the drive shaft is further mechanically coupled to the second mixing arm and the vacuum creation system such that rotation of the drive shaft forces the first and second mixing arms to move and the vacuum creation system to be actuated.

3. The mixer of claim 1, wherein the drive shaft is mechanically coupled with the first mixing arm via a worm and worm gear, and wherein the worm and worm gear are configured cause the first mixing arm to rotate at a slower rate than a rate at which the drive shaft rotates.

4. The mixer of claim 1, further comprising a pressure-regulating valve that is configured to be automatically actuated to prevent the vacuum creation system from drawing additional gas from the container when a set vacuum level is obtained in the container.

5. A bone cement mixer comprising:
a container configured to hold an amount of bone cement;
a drive shaft coupled to the container;
a mixing arm coupled to the container and configured to mix the bone cement when the bone cement is disposed within the container; and
a vacuum pump configured to draw gas from within the container to form at least a partial vacuum within the container,
wherein the drive shaft is mechanically coupled to the mixing arm and the vacuum pump such that rotation of the drive shaft forces the mixing arm to move and the vacuum pump to be actuated.

6. The mixer of claim 5, wherein the drive shaft mechanically couples with the mixing arm via a worm drive.

7. The mixer of claim 6, wherein the worm drive is configured to cause the mixing arm to rotate at a slower rate than a rate at which the drive shaft rotates.

8. The mixer of claim 5, further comprising a filter, wherein the filter is configured to filter gas that is drawn by the vacuum pump from within the container.

9. The mixer of claim 5, further comprising a powered rotary device that is coupled to the container and that is configured to turn the drive shaft.

10. The mixer of claim 9, wherein the rotary device comprises at least one of a drill and a reamer-driver that is coupled to the drive shaft.

11. The mixer of claim 9, wherein the rotary device comprises a motor and a battery that are coupled to the container.

12. The mixer of claim 5, further comprising a pressure-regulating valve that is configured to be actuated to prevent the vacuum pump from drawing additional gas from the container when a set vacuum level is obtained in the container.

13. The mixer of claim 5, further comprising a one-way check valve that is configured to prevent gas from entering into the container through the vacuum pump when the container is sealed.

14. The mixer of claim 5, further comprising a clutch that is configured to allow a ratio of a rate at which the mixing arm rotates and a rate at which a portion of the drive shaft rotates to vary from each other.

15. The mixer of claim 5, further comprising a pressure-relief valve that is configured to selectively release the partial vacuum within the container when the pressure-relief valve is actuated.

16. A bone cement mixer comprising:
a container configured to hold an amount of bone cement;
a drive shaft coupled to the container;
a mixing arm coupled to the container and configured to mix the bone cement when the bone cement is disposed within the container;
a vacuum pump configured to draw gas from within the container to form at least a partial vacuum within the container;
a pressure-regulating valve that is configured to be actuated to prevent the vacuum pump from drawing additional gas from the container when a set vacuum level is obtained in the container; and
a one-way check valve that is configured to prevent gas from entering into the container through the vacuum pump when the container is sealed,
wherein the drive shaft is mechanically coupled to the mixing arm and the vacuum pump such that rotation of the drive shaft forces the mixing arm to move and the vacuum pump to be actuated.

17. The mixer of claim 16, further comprising a drive mechanism that is configured to convert rotational force of the drive shaft into rotational force of the mixing arm, and wherein drive mechanism is configured to cause the mixing arm to rotate at a slower rate than a rate at which the drive shaft rotates.

18. The mixer of claim 16, further comprising a powered rotary device that is coupled to the container and that is configured to turn the drive shaft, wherein the rotary device comprises at least one of a drill and a reamer-driver that is coupled to the drive shaft.

19. The mixer of claim 16, further comprising a filter, wherein the filter is configured to filter gas that is drawn by the vacuum pump from within the container.

20. The mixer of claim 16, further comprising a torque clutch that is configured to allow a ratio of a rate at which the mixing arm rotates and a rate at which a portion of the drive shaft rotates to vary from each other.
